(19) United States
(12) Patent Application Publication
(10) Pub. No.: US 2010/0286105 A1
(43) Pub. Date: Nov. 11, 2010

(54) METHODS OF TREATING HYPERANDROGENISM AND CONDITIONS ASSOCIATED THEREWITH BY ADMINISTERING A FATTY ACID ESTER OF AN ESTROGEN OR AN ESTROGEN DERIVATIVE

(75) Inventor: Bindu Tejura, Cambridge, MA (US)

Correspondence Address: HOXIE & ASSOCIATES LLC, 75 MAIN STREET , SUITE 301, MILLBURN, NJ, 07041, US

(73) Assignee: Bindu Tejura, Cambridge, MA (US)

(21) Appl. No.: 12/443,451

(22) PCT Filed: Sep. 28, 2007
(86) PCT No.: PCT/US2007/020932

§ 371 Date: Mar. 27, 2009

Related U.S. Application Data
(60) Provisional application No. 60/848,561, filed on Sep. 29, 2006.

(51) Int. Cl. A61K 31/566; A61P 5/26; A61P 35/00; A61P 5/50; A61P 17/10; A61P 3/06; A61P 3/10; A61P 3/04; A61P 17/14; C07J 1/00

(52) U.S. Cl. 514/178; 552/625

(57) ABSTRACT

Compositions and methods for treating hyperandrogenism and conditions associated therewith, including polycystic ovary syndrome are disclosed. The invention is directed to methods for treating hyperandrogenism and conditions associated therewith, in a subject comprising administering a fatty acid ester of an estrogen or an estrogen derivative. Furthermore the invention is directed to compositions for treating hyperandrogenism and conditions associated with hyperandrogenism, including polycystic ovary syndrome, comprising a fatty acid ester of an estrogen or an estrogen derivative.

### RELATED APPLICATIONS

This invention claims benefit, under 35 U.S.C. §119(e), of U.S. provisional application No. 60/848,561, filed on Sep. 29, 2006, which is herein incorporated by reference in its entirety.

### FIELD OF THE INVENTION

This invention relates generally to methods and pharmaceutical compositions for treating hyperandrogenism and conditions associated with hyperandrogenism, including polycystic ovary syndrome. More particularly, the invention is directed to methods of treating hyperandrogenism and conditions associated therewith, including polycystic ovary syndrome, comprising administering a fatty acid ester of an estrogen or an estrogen derivative. Furthermore, the invention is directed to pharmaceutical compositions comprising a fatty acid ester of an estrogen or an estrogen derivative for treating hyperandrogenism and conditions associated therewith.

### BACKGROUND

Elevated levels of testosterone and other androgens, i.e., hyperandrogenism, have been linked to a number of conditions. These include ovarian, breast, endometrial and prostate cancers; polycystic ovary syndrome (PCOS); hirsutism; hyperplasia, such as congenital adrenal hyperplasia; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; overweight; obesity, excess body and facial hair; hypertension; Cushing Syndrome; HAIR-AN (hyperandrogenism, insulin resistance and acanthosis nigricans) Syndrome; virilism; and alopecia. Current treatments of hyperandrogenism and conditions associated therewith usually include suppression of testosterone or androgen production through surgery, such as prostatectomy, or administration of androgen or hormonal ablation agents. However, success of current treatments varies widely among patients.

For example, PCOS, also known as Stein-Leventhal syndrome, is a hormone-related disorder affecting about 4% to 7% of reproductive-aged women. Australian Family Physician, Vol. 34, No. 3 March 2005. Although the exact cause of PCOS is still unknown, PCOS is generally characterized by (1) anovulation, i.e., lack of ovulation or erratic or infrequent ovulation, as well as (2) hyperandrogenism. Women diagnosed with PCOS also can exhibit irregular or lack of menstrual periods, an increased risk of miscarriage, as well as, other conditions that are also associated with hyperandrogenism.

Currently, treatment of PCOS is not directed at addressing the underlying cause(s), but is instead largely directed to the treatment of the symptoms and conditions associated with PCOS. For instance, women who are diagnosed with PCOS and suffer from overweight, obesity, or impaired glucose tolerance are encouraged to make lifestyle changes that include dietary modifications, such as reduced caloric intake, and an increased physical activity. However, such lifestyle changes are often unsuccessful due to lack of patient compliance.

Furthermore, other methods of treating PCOS have shown limitations. For example women diagnosed with PCOS have been subjected to ovulation induction treatments, follicle stimulating hormone (FSH) injections and laparoscopic ovarian diathermy. However, success of such treatments in women diagnosed with PCOS is highly variable.

Oral contraceptive pills have also been used for the treatment of PCOS. However, the use of oral contraceptives to treat PCOS is subject to debate due to potential adverse effects on insulin resistance, glucose tolerance, vascular reactivity and coagulability. Therefore, there exists a need for additional ways to treat hyperandrogenism, as well as, conditions associated with hyperandrogenism including, but not limited to, PCOS.

### SUMMARY

Since it is of interest to provide satisfactory new approaches for the treatment of hyperandrogenism and conditions associated therewith, the present invention relates generally to pharmaceutical compositions and methods for treating hyperandrogenism, as well as, conditions associated with hyperandrogenism, in a subject, such as a human.

More particularly, the invention is directed to methods for treating hyperandrogenism and conditions associated with hyperandrogenism, such as polycystic ovary syndrome, in a subject comprising administering a therapeutically effective amount a fatty acid ester of an estrogen or an estrogen derivative. Furthermore the invention is directed to pharmaceutical compositions comprising a fatty acid ester of an estrogen or an estrogen derivative for the treatment of hyperandrogenism and conditions associated with hyperandrogenism, such as polycystic ovary syndrome.

Therefore, the invention comprises the following scopes:
Scope 1) A method of treating hyperandrogenism and conditions relating to hyperandrogenism in a subject comprising administering a fatty acid ester of an estrogen or an estrogen derivative.
Scope 2) The method of Scope 1, wherein the estrogen comprises estrone, diethylstilbestrol, estriol, estradiol or ethinyl estradiol.
Scope 3) The method of Scope 1, wherein the estrogen comprises estrone.
Scope 4) The method of Scope 1, wherein the estrogen derivative comprises 2-hydroxyestrone or 2-hydroxy-β-estradiol.
Scope 5) The method of Scope 1, wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid.
Scope 6) The method of Scope 1, wherein the fatty acid comprises an oleic acid.
Scope 7) The method of Scope 1, wherein the fatty acid includes an acyl group; and wherein when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 8) The method of Scope 1, wherein the fatty acid includes an acyl group; wherein the estrogen comprises estrone, diethylstilbestrol, estriol or ethinyl estradiol; and wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid; and with the proviso that, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 9) The method of Scope 1, wherein the fatty acid ester of an estrogen or an estrogen derivative is a fatty acid monoester.
Scope 10) The method of Scope 1, wherein the fatty acid ester of an estrogen or an estrogen derivative comprises oleoyl-estrone.
Scope 11) The method of Scope 1, wherein the fatty acid ester of an estrogen or an estrogen derivative is administered orally.
Scope 12) The method of Scope 1, wherein the fatty acid ester of an estrogen or an estrogen derivative is administered in an amount of about 0.000005 mg/kg/day to about 20 mg/kg/day.
Scope 13) The method of Scope 1, wherein the fatty acid ester of an estrogen or an estrogen derivative is administered in an amount of about 0.00005 mg/kg/day to about 10 mg/kg/day.
Scope 14) The method of Scope 1, wherein the fatty acid ester of estrogen or an estrogen derivative is administered in an amount of about 0.0005 mg/kg/day to about 2 mg/kg/day.
Scope 15) The method of Scope 1, wherein the fatty acid ester of an estrogen or an estrogen derivative is administered in an amount of about 0.001 mg/kg/day to about 1 mg/kg/day.
Scope 16) The method of Scope 1, wherein the fatty acid ester of an estrogen or an estrogen derivative is administered in the form of a pharmaceutical composition.
Scope 17) The method of scope 16, wherein the pharmaceutical composition comprises at least one pharmaceutically-acceptable excipient.
Scope 18) The method of scope 16, wherein the fatty acid ester of an estrogen or an estrogen derivative comprises about 0.01% to about 99% by weight of the pharmaceutical composition.
Scope 19) The method of scope 16, wherein the fatty acid ester of an estrogen or an estrogen derivative comprises about 0.1% to about 45% by weight of the pharmaceutical composition.
Scope 20) The method of scope 16, wherein the fatty acid ester of an estrogen or an estrogen derivative comprises about 1% to about 15% by weight of the pharmaceutical composition.
Scope 21) The method of scope 16, wherein the pharmaceutical composition further comprises an oil.
Scope 22) The method of scope 21, wherein the oil comprises a triglyceride.
Scope 23) The method of scope 21, wherein the oil comprises an oil derived from a plant.
Scope 24) The method of scope 21, wherein the oil comprises canola oil, safflower oil, soybean oil, corn oil, olive oil, sesame oil, rapeseed oil, sunflower oil, rice oil, palm oil, cottonseed oil, linseed oil, tung oil, oiticica oil, castor oil, perilla oil, coconut oil, Labrasol®, Labrafil® M 125CS, Labrafac® CC, Captex® 300-6C, Captex® 300 EP, tallow, lard, stearic acid, fish oil, fish-liver oil, oleic acid, sperm oil, petroleum oil, or combinations thereof.
Scope 25) The method of scope 21, wherein the oil comprises safflower oil, canola oil or soybean oil.
Scope 26) The method of scope 21, wherein the oil comprises about 25% to about 99% by weight of the pharmaceutical composition.
Scope 27) The method of scope 21, wherein the oil comprises about 75% to about 99% by weight of the pharmaceutical composition.
Scope 28) The method of scope 21, wherein the oil comprises about 80% to about 99% by weight of the pharmaceutical composition.
Scope 29) The method of scope 16, wherein the pharmaceutical composition is contained in a capsule.
Scope 30) The method of scope 1, wherein the conditions associated with hyperandrogenism comprise ovarian, breast, endometrial and prostate cancer, as well as other conditions such as, polycystic ovary syndrome; Cushing Syndrome; HAIR-AN (hyperandrogenism, insulin resistance and acanthosis nigricans) Syndrome; hirsutism; hyperplasia, such as congenital adrenal hyperplasia; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; overweight; obesity, excess body and facial hair; and alopecia.
Scope 31) A method for treating hyperandrogenism comprising orally administering a pharmaceutical composition comprising a therapeutically effective amount of oleoyl-estrone and a pharmaceutically-acceptable excipient.
Scope 32) A method for treating polycystic ovary syndrome comprising administering a therapeutically effective amount of a fatty acid ester of an estrogen or an estrogen derivative.
Scope 33) The method of scope 32, wherein the estrogen comprises estrone, diethylstilbestrol, estriol, estradiol or ethinyl estradiol.
Scope 34) The method of scope 32, wherein the estrogen comprises estrone.
Scope 35) The method of scope 32, wherein the estrogen derivative comprises 2-hydroxyestrone or 2-hydroxy-β-estradiol.
Scope 36) The method of scope 32, wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid.
Scope 37) The method of scope 32, wherein the fatty acid comprises an oleic acid.
Scope 38) The method of scope 32, wherein the fatty acid includes an acyl group; and wherein when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 39) The method of scope 32 wherein the fatty acid includes an acyl group; wherein the estrogen comprises estrone, diethylstilbestrol, estriol or ethinyl estradiol; and wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid; and with the proviso that, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 40) The method of scope 32, wherein the fatty acid ester of an estrogen or an estrogen derivative is a fatty acid monoester.
Scope 41) The method of scope 32, wherein the fatty acid ester of an estrogen or an estrogen derivative comprises oleoyl-estrone.
Scope 42) The method of scope 32, wherein the fatty acid ester of an estrogen or an estrogen derivative is administered orally.
Scope 43) The method of scope 32, wherein the fatty acid ester of an estrogen or an estrogen derivative is administered in an amount of about 0.000005 mg/kg/day to about 20 mg/kg/day.
Scope 44) The method of scope 32, wherein the fatty acid ester of an estrogen or an estrogen derivative is administered in an amount of about 0.00005 mg/kg/day to about 10 mg/kg/day.
Scope 45) The method of scope 32, wherein the fatty acid ester of estrogen or an estrogen derivative is administered in an amount of about 0.0005 mg/kg/day to about 2 mg/kg/day.
Scope 46) The method of scope 32, wherein the fatty acid ester of an estrogen or an estrogen derivative is administered in an amount of about 0.001 mg/kg/day to about 1 mg/kg/day.
Scope 47) The method of scope 32, wherein the fatty acid ester of an estrogen or an estrogen derivative is administered in the form of a pharmaceutical composition.
Scope 48) The method of scope 47, wherein the pharmaceutical composition comprises at least one pharmaceutically-acceptable excipient.
Scope 49) The method of scope 47, wherein the fatty acid ester of an estrogen or an estrogen derivative comprises about 0.01% to about 99% by weight of the pharmaceutical composition.
Scope 50) The method of scope 47, wherein the fatty acid ester of an estrogen or an estrogen derivative comprises about 0.1% to about 45% by weight of the pharmaceutical composition.
Scope 51) The method of scope 47, wherein the fatty acid ester of an estrogen or an estrogen derivative comprises about 1% to about 15% by weight of the pharmaceutical composition.
Scope 52) The method of scope 47, wherein the pharmaceutical composition further comprises an oil.
Scope 53) The method of scope 52, wherein the oil comprises a triglyceride.
Scope 54) The method of scope 52, wherein the oil comprises an oil derived from a plant.
Scope 55) The method of scope 52, wherein the oil comprises canola oil, safflower oil, soybean oil, corn oil, olive oil, sesame oil, rapeseed oil, sunflower oil, rice oil, palm oil, cottonseed oil, linseed oil, tung oil, oiticica oil, castor oil, perilla oil, coconut oil, Labrasol®, Labrafil® M 125CS, Labrafac® CC, Captex® 300-6C, Captex® 300 EP, tallow, lard, stearic acid, fish oil, fish-liver oil, oleic acid, sperm oil, petroleum oil, or combinations thereof.
Scope 56) The method of scope 52, wherein the oil comprises safflower oil, canola oil or soybean oil.
Scope 57) The method of scope 52, wherein the oil comprises about 25% to about 99% by weight of the pharmaceutical composition.
Scope 58) The method of scope 52, wherein the oil comprises about 75% to about 99% by weight of the pharmaceutical composition.
Scope 59) The method of scope 52, wherein the oil comprises about 80% to about 99% by weight of the pharmaceutical composition.
Scope 60) The method of scope 47, wherein the pharmaceutical composition is contained in a capsule.
Scope 61) The method of scope 32, wherein treating PCOS comprises treating the conditions associated with polycystic ovary syndrome comprise anovulation; oligoamenorrhea; amenorrhea; hirsutism; infertility; increased risk of miscarriage; miscarriage, ovarian cysts; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; hypertension; virilism; Cushing Syndrome; overweight; obesity; excess body and facial hair; and alopecia.
Scope 62) A method for treating polycystic ovary syndrome comprising orally administering a pharmaceutical composition comprising a therapeutically effective amount of oleoyl-estrone and a pharmaceutically-acceptable excipient.
Scope 63) A pharmaceutical composition for treating hyperandrogenism and conditions associated with hyperandrogenism comprising a therapeutically effective amount of a fatty acid ester of an estrogen or an estrogen derivative.
Scope 64) The composition of scope 63, wherein the estrogen comprises estrone, diethylstilbestrol, estriol, estradiol or ethinyl estradiol.
Scope 65) The composition of scope 63, wherein the estrogen comprises estrone.
Scope 66) The composition of scope 63, wherein the estrogen derivative comprises 2-hydroxyestrone or 2-hydroxy-β-estradiol.
Scope 67) The composition of scope 63, wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid.
Scope 68) The composition of scope 63, wherein the fatty acid comprises an oleic acid.
Scope 69) The composition of scope 63, wherein the fatty acid includes an acyl group; and wherein when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 70) The composition of scope 63, wherein the fatty acid includes an acyl group; wherein the estrogen comprises estrone, diethylstilbestrol, estriol or ethinyl estradiol; and wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid; and with the proviso that, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 71) The composition of scope 63, wherein the fatty acid comprises a fatty acid monoester.
Scope 72) The composition of scope 63, wherein the fatty acid ester of an estrogen or an estrogen derivative comprises oleoyl-estrone.
Scope 73) The composition of scope 72, wherein the pharmaceutical composition further comprises at least one pharmaceutically acceptable excipient.
Scope 74) The composition of scope 72, wherein the composition is contained in a capsule.
Scope 75) The composition of scope 72, wherein the fatty acid ester of an estrogen or an estrogen derivative comprises an amount of about 0.000005 mg/kg/day to about 20 mg/kg/day.
Scope 76) The composition of scope 72, wherein the fatty acid ester of an estrogen or an estrogen derivative comprises an amount of about 0.00005 mg/kg/day to about 10 mg/kg/day.
Scope 77) The composition of scope 72, wherein the fatty acid ester of an estrogen or an estrogen derivative comprises an amount of about 0.0005 mg/kg/day to about 2 mg/kg/day.
Scope 78) The composition of scope 72, wherein the fatty acid ester of an estrogen or an estrogen derivative comprises an amount of about 0.001 mg/kg/day to about 1 mg/kg/day.
Scope 79) The composition of scope 72, wherein the fatty acid ester of an estrogen or an estrogen derivative comprises about 0.01% to about 99% by weight of the composition.
Scope 80) The composition of scope 72, wherein the fatty acid ester of an estrogen or an estrogen derivative comprises about 0.1% to about 45% by weight of the composition.
Scope 81) The composition of scope 72, wherein the fatty acid ester of an estrogen or an estrogen derivative comprises about 1% to about 15% by weight of the composition.
Scope 82) The composition of scope 72, wherein the composition comprises an oil.
Scope 83) The composition of scope 82, wherein the oil comprises a triglyceride.
Scope 84) The composition of scope 82, wherein the oil comprises an oil derived from a plant.
Scope 85) The composition of scope 82, wherein the oil comprises, canola oil, safflower oil, soybean oil, corn oil, olive oil, sesame oil, rapeseed oil, sunflower oil, rice oil, palm oil, cottonseed oil, linseed oil, tung oil, oiticica oil, castor oil, perilla oil, coconut oil, Labrasol®, Labrafil® M 125CS, Labrafac® CC, Captex® 300-6C, Captex® 300 EP, tallow, lard, stearic acid, fish oil, fish-liver oil, oleic acid, sperm oil, petroleum oil, or combinations thereof.
Scope 86) The composition of scope 82, wherein the oil comprises safflower oil, canola oil or soybean oil.
Scope 87) The composition of scope 82, wherein the oil comprises about 25% to about 99% by weight of the composition.
Scope 88) The composition of scope 82, wherein the oil comprises about 75% to about 99% by weight of the composition.
Scope 89) The composition of scope 82, wherein the oil comprises about 80% to about 99% by weight of the composition.
Scope 90) A pharmaceutical composition for treating hyperandrogenism and the conditions associated with hyperandrogenism comprising oleoyl-estrone and an oil, wherein the composition is contained in a capsule.
Scope 91) A composition for treating polycystic ovary syndrome comprising a therapeutically effective amount of a fatty acid ester of an estrogen or an estrogen derivative.
Scope 92) The composition of scope 91, wherein the estrogen comprises estrone, diethylstilbestrol, estriol, estradiol or ethinyl estradiol.
Scope 93) The composition of scope 91, wherein the estrogen comprises estrone.
Scope 94) The composition of scope 91, wherein the estrogen derivative comprises 2-hydroxyestrone or 2-hydroxy-β-estradiol.
Scope 95) The composition of scope 91, wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid.
Scope 96) The composition of scope 91, wherein the fatty acid comprises an oleic acid.

Scope 97) The composition of scope 91, wherein the fatty acid includes an acyl group; and wherein when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 98) The composition of scope 91, wherein the fatty acid includes an acyl group; wherein the estrogen comprises estrone, diethylstilbestrol, estriol or ethinyl estradiol; and wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid; and with the proviso that, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.
Scope 99) The composition of scope 91, wherein the fatty acid comprises a fatty acid monoester.
Scope 100) The composition of scope 91, wherein the fatty acid ester of an estrogen or an estrogen derivative comprises oleoyl-estrone.
Scope 101) The composition of scope 91, wherein the pharmaceutical composition further comprises at least one pharmaceutically acceptable excipient.
Scope 102) The composition of scope 91, wherein the composition is contained in a capsule.
Scope 103) The composition of scope 91, wherein the fatty acid ester of an estrogen or an estrogen derivative comprises an amount of about 0.000005 mg/kg/day to about 20 mg/kg/day.
Scope 104) The composition of scope 91, wherein the fatty acid ester of an estrogen or an estrogen derivative comprises an amount of about 0.00005 mg/kg/day to about 10 mg/kg/day.
Scope 105) The composition of scope 91, wherein the fatty acid ester of an estrogen or an estrogen derivative comprises an amount of about 0.0005 mg/kg/day to about 2 mg/kg/day.
Scope 106) The composition of scope 91, wherein the fatty acid ester of an estrogen or an estrogen derivative comprises an amount of about 0.001 mg/kg/day to about 1 mg/kg/day.
Scope 107) The composition of scope 91, wherein the fatty acid ester of an estrogen or an estrogen derivative comprises about 0.01% to about 99% by weight of the composition.
Scope 108) The composition of scope 91, wherein the fatty acid ester of an estrogen or an estrogen derivative comprises about 0.1% to about 45% by weight of the composition.
Scope 109) The composition of scope 91, wherein the fatty acid ester of an estrogen or an estrogen derivative comprises about 1% to about 15% by weight of the composition.
Scope 110) The composition of scope 91, wherein the composition comprises an oil.
Scope 111) The composition of scope 110, wherein the oil comprises a triglyceride.
Scope 112) The composition of scope 110, wherein the oil comprises an oil derived from a plant.
Scope 113) The composition of scope 110, wherein the oil comprises, canola oil, safflower oil, soybean oil, corn oil, olive oil, sesame oil, rapeseed oil, sunflower oil, rice oil, palm oil, cottonseed oil, linseed oil, tung oil, oiticica oil, castor oil, perilla oil, coconut oil, Labrasol®, Labrafil® M 125CS, Labrafac® CC, Captex® 300-6C, Captex® 300 EP, tallow, lard, stearic acid, fish oil, fish-liver oil, oleic acid, sperm oil, petroleum oil, or combinations thereof.
Scope 114) The composition of scope 110, wherein the oil comprises safflower oil, canola oil or soybean oil.
Scope 115) The composition of scope 110, wherein the oil comprises about 25% to about 99% by weight of the composition.
Scope 116) The composition of scope 110, wherein the oil comprises about 75% to about 99% by weight of the composition.
Scope 117) The composition of scope 110, wherein the oil comprises about 80% to about 99% by weight of the composition.
Scope 118) A pharmaceutical composition for treating polycystic ovary syndrome comprising oleoyl-estrone and an oil, wherein the composition is contained in a capsule.
Scope 119) An article of manufacture comprising packaging material and a pharmaceutical composition described in any one of scopes 90-118.
Scope 120) An article of manufacture according to scope 119, further comprising printed instructions regarding the use or administration of the pharmaceutical composition.
Scope 121) An article of manufacture according to scope 119 or 120, wherein the instruction suggests a dosing regiment for the treatment of hyperandrogenism and conditions relating to hyperandrogenism in a subject.
Scope 122) An article of manufacture according to any one of scopes 119-121, wherein said hyperandrogenism and conditions associated with hyperandrogenism is polycystic ovary syndrome.
Scope 123) A method of modulating the levels of androgens in a subject in need thereof comprising administering a fatty acid ester of an estrogen or an estrogen derivative described in any one of scopes 1-118.
Scope 124) Use of a fatty acid ester of an estrogen or an estrogen derivative described in any one of scopes 1-123 in the manufacture of a medicament for the treatment of hyperandrogenism and conditions associated with hyperandrogenism.
Scope 125) Use according to scope 124, wherein the conditions associated with hyperandrogenism comprise ovarian, breast, endometrial and prostate cancer, as well as other conditions such as, polycystic ovary syndrome; Cushing Syndrome; HAIR-AN (hyperandrogenism, insulin resistance and acanthosis nigricans) Syndrome; hirsutism; hyperplasia, such as congenital adrenal hyperplasia; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; overweight; obesity, excess body and facial hair; and alopecia.
Scope 126) Use according to scope 124, wherein the conditions associated with hyperandrogenism is polycystic ovary syndrome.

In this specification, the term “anovulation” refers to the absence of ovulation or erratic or infrequent ovulation.

The term “estrogen” refers to the substances tending to promote estrus and stimulate the development of female secondary sex characteristics. This term comprises natural, semisynthetic and synthetic estrogens, both steroidal and nonsteroidal, such as estrone, diethylstilbestrol, estriol, estradiol and ethinyl estradiol.

The term “estrogen derivative” refers to a compound that is derived from estrogen and usually maintains its general structure. Estrogen and estrogen derivatives are substances that induce biological responses linked to the stimulation of estrogen receptors and other biological systems that result in biological actions similar to those of estradiol and estrone.

In this specification, the phrase “fatty acid” refers to the carboxylic acids that are components of natural fats, such as, without limitation, oleic, linoleic, linolenic, stearic, palmitic, palmitoleic, arachidonic, eicosenoic, docosenoic, and tetracosenoic acids.

The term “fatty acid ester” or “fatty acid ester of an estrogen or an estrogen derivative” refers to any compound containing one or more fatty acids bonded to an estrogen or an estrogen derivative molecule.

As used herein the term “hyperandrogenism” is used to describe a condition involving excessive secretion of androgens, including without limitation, testosterone by organs such as the adrenal cortex, ovaries or testes.

As used herein, the terms “subject,” “patient” and “animal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) or a primate (e.g., a monkey, chimpanzee and a human), and more preferably a human. In preferred embodiments the subject is a woman. In one embodiment, the subject is a non-human animal such as a farm animal (e.g., a horse, cow, pig or sheep), or a pet (e.g., a dog, cat, guinea pig or rabbit).

The phrase “therapeutically effective amount” refers to that amount of the fatty acid ester of an estrogen or an estrogen derivative that is sufficient to provide a therapeutic benefit in the treatment or management of a condition, such as hyperandrogenism and conditions associated with hyperandrogenism, including PCOS, or to minimize hyperandrogenism and conditions associated with hyperandrogenism, including PCOS. Further, a therapeutically effective amount with respect to a fatty acid ester of an estrogen or an estrogen derivative means that amount of therapeutic agent alone, or in combination with other therapies, that provides a therapeutic benefit in the treatment or management a condition, such as hyperandrogenism and conditions associated with hyperandrogenism, including PCOS. Used in connection with an amount of a fatty acid ester of an estrogen or an estrogen derivative, the term can encompass an amount that improves overall therapy, reduces or avoids symptoms or causes of a condition, such as hyperandrogenism and conditions associated with hyperandrogenism, including PCOS, or enhances the therapeutic efficacy of or synergies with another therapeutic agent.

As used herein, the terms “treat”, “treating” and “treatment” refer to the elimination, reduction or amelioration of a condition, such as hyperandrogenism, as well as, conditions associated with hyperandrogenism, such as PCOS. Furthermore, the treatment of PCOS does not only include the treatment of the PCOS but also the treatment of any conditions or syndromes associated with PCOS.

The terms “prevent(s)”, “prevented”, “preventing” or “prevention” are used herein interchangeably and refer to any prevention or any contribution to the prevention of a condition, such as hyperandrogenism, as well as, conditions associated with hyperandrogenism, such as PCOS in a patient or the development of a condition if none has occurred in a patient which may be predisposed to such condition but has not yet been afflicted with or diagnosed as having such condition.

The term “modulating” refers to the change in the level of androgen, such as testosterone, in a subject brought about, in whole or in part and directly or indirectly, by the administration of a fatty acid ester of an estrogen or an estrogen derivative of the current invention.

### DETAILED DESCRIPTION

As noted above, the present invention relates to methods and pharmaceutical compositions for the treatment of hyperandrogenism and conditions associated with hyperandrogenism. For example, the present invention is directed to methods of treating or preventing hyperandrogenism, by administering to a subject, preferably a human, a therapeutically effective amount of a fatty acid ester of an estrogen or an estrogen derivative. In certain embodiments, the fatty acid ester of an estrogen or an estrogen derivative is oleoyl-estrone. Additionally, the present invention is directed to a pharmaceutical composition comprising a therapeutically effective amount of a fatty acid ester of estrogen or an estrogen derivative for treating or preventing hyperandrogenism in a subject. In certain embodiments, the fatty acid ester of an estrogen or an estrogen derivative of the pharmaceutical composition is oleoyl-estrone.

Conditions associated with hyperandrogenism include, but are not limited to, ovarian, breast, endometrial and prostate cancers; PCOS; hirsutism; hyperplasia, such as congenital adrenal hyperplasia; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; overweight; obesity, excess body and facial hair; hypertension; Cushing Syndrome; HAIR-AN (hyperandrogenism, insulin resistance and acanthosis nigricans) Syndrome; virilism; and alopecia.

Additionally, the present invention is also directed to methods of treating or preventing PCOS, by administering to a subject, preferably a human, a therapeutically effective amount of a fatty acid ester of an estrogen or an estrogen derivative. In certain embodiments, the fatty acid ester of an estrogen or an estrogen derivative is oleoyl-estrone. The present invention is also directed to a pharmaceutical composition for treating or preventing PCOS comprising a therapeutically effective amount of a fatty acid ester of estrogen or an estrogen derivative. In certain embodiments, the fatty acid ester of an estrogen or an estrogen derivative is oleoyl-estrone.

The methods of treating or preventing PCOS also include methods of treating or preventing any conditions associated with PCOS. Conditions associated with PCOS can also include the same conditions that are associated with hyperandrogenism. Conditions associated with PCOS include, but are not limited to, anovulation; oligoamenorrhea; amenorrhea; hirsutism; infertility; increased risk of miscarriage; miscarriage; ovarian cysts; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; overweight; obesity, excess body and facial hair; and alopecia.

### Fatty Acid Esters of an Estrogen or an Estrogen Derivative

In some embodiments, the estrogen of the fatty acid ester of an estrogen or an estrogen derivative comprises estrone, (i.e. 3-hydroxyestra-1,3,5(10)-trien-17-one); diethylstilbestrol, (i.e. 4,4′-(1,2-diethyl-1,2-ethenediyl)-bisphenol); estriol, (i.e. estra-1,3,5(10)triene-3,16,17-triol), ethinyl estradiol, (i.e. 19-nor-17a-pregna-1,3,5(10)-trien-20-3,17-diol); or estradiol. The estrogen preferably comprises an estrone. Further, in some embodiments, the estrogen derivative of the fatty acid ester of an estrogen or an estrogen derivative preferably comprises 2-hydroxyestrone or 2-hydroxy-β-estradiol.

In some embodiments, the fatty acid can comprise, for instance, oleic acid, linoleic acid, linolenic acid, stearic acid, palmitic acid, palmitoleic acid, arachidonic acid, eicosenoic acid, docosenoic acid, tetracosenoic acid or any other aliphatic acid with a straight, branched, substituted, saturated, or unsaturated chain. Preferably, the fatty acid comprises an oleic acid.

In some embodiments, the fatty acid includes an acyl group and the estrogen is steroidal and has a steroid ring system with a C-3 position and a hydroxyl group at the C-3 position. In these embodiments, the acyl group of the fatty acid is attached to the hydroxyl group at the C-3 position of the steroid ring system in the fatty acid ester.

In other embodiments, the fatty acid includes an acyl group, wherein the estrogen comprises estrone, diethylstilbestrol, estriol or ethinyl estradiol, and wherein the fatty acid comprises an oleic acid, linoleic acid, linolenic acid, stearic acid, palmitic acid, palmitoleic acid, arachidonic acid, eicosenoic acid, docosenoic acid, or tetracosenoic acid; and with the proviso that, when the estrogen is steroidal and has a steroid ring system with a C-3 position and a hydroxyl group at the C-3 position, the acyl group of the fatty acid is attached to the hydroxyl group at the C-3 position of the steroid ring system in the fatty acid ester.

The fatty acid ester of estrogen or an estrogen derivative preferably comprises a fatty acid monoester, such as, for example, estrone monooleate (“oleoyl-estrone”), diethylstilbestrol monooleate, estrone monoeicosenoate or diethylstilbestrol monoeicosenoate.

The chemical name of oleoyl-estrone is [3(Z)]-3-[(1-oxo-9-octadecenyl)oxy]-estra-1,3,5(10)-trien-17-one and the chemical formula of oleoyl-estrone is:

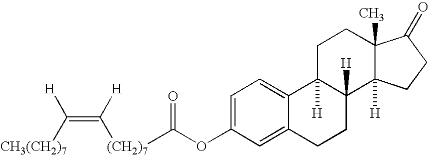

The dose of the fatty acid ester of an estrogen or an estrogen derivative is dependent upon a number of factors, including, the nature of the severity of the condition to be treated, the route of administration and the age, weight, body-mass-index (BMI) and response of the individual patient. For example, the observed levels of the compound following administration are lower in patients having a high BMI, meaning that such patients may need a higher dose than patients having a lower BMI. If desired, the dosage may be adjusted based on the observed levels of compound in the individual patient and the patient's response to treatment. The dose of the fatty acid ester of an estrogen or an estrogen derivative can generally range from about 0.000005 mg/kg/day to about 20 mg/kg/day.

For the treatment of hyperandrogenism and conditions associated with hyperandrogenism, the therapeutically effective amount of the fatty acid ester of an estrogen or an estrogen derivative comprises an amount of about 0.00005 mg/kg/day to about 10 mg/kg/day or the therapeutically effective amount of the fatty acid ester of an estrogen or an estrogen derivative comprises an amount of about 0.0005 mg/kg/day to about 2 mg/kg/day. Preferably, the therapeutically effective amount of the fatty acid ester of an estrogen or an estrogen derivative comprises an amount of about 0.001 mg/kg/day to about 1 mg/kg/day.

Preferably, for the treatment of PCOS, the therapeutically effective amount of the fatty acid ester of an estrogen or an estrogen derivative comprises an amount of about 0.00005 mg/kg/day to about 10 mg/kg/day or the therapeutically effective amount of the fatty acid ester of an estrogen or an estrogen derivative comprises an amount of about 0.0005 mg/kg/day to about 2 mg/kg/day. More preferably, the therapeutically effective amount of the fatty acid ester of an estrogen or an estrogen derivative comprises an amount of about 0.001 mg/kg/day to about 1 mg/kg/day.

The fatty acid ester of an estrogen or an estrogen derivative can be administered as a single daily dose or multiple daily doses or administered in doses over various other time periods. The daily dose can be between about 0.001 mg/day to about 1000 mg/day.

For the treatment of conditions associated with hyperandrogenism, the therapeutically effective amount of the fatty acid ester of an estrogen or an estrogen derivative comprises an amount of about 0.01 mg/day to about 500 mg/day or the therapeutically effective amount of the fatty acid ester of an estrogen or an estrogen derivative comprises an amount of about 0.1 mg/day to about 100 mg/day. Preferably, the therapeutically effective amount of the fatty acid ester of an estrogen or an estrogen derivative comprises an amount of about 1 mg/day to about 50 mg/day. In some cases it may be necessary to use dosages outside of these ranges.

For the treatment of PCOS, the therapeutically effective amount of the fatty acid ester of an estrogen or an estrogen derivative comprises an amount of about 0.01 mg/day to about 500 mg/day or the therapeutically effective amount of the fatty acid ester of an estrogen or an estrogen derivative comprises an amount of about 0.1 mg/day to about 100 mg/day. Preferably, the therapeutically effective amount of the fatty acid ester of an estrogen or an estrogen derivative comprises an amount of about 1 mg/day to about 50 mg/day. In some cases it may be necessary to use dosages outside of these ranges.

### Pharmaceutical Compositions

In certain embodiments of the present invention, the fatty acid ester of an estrogen or an estrogen derivative is incorporated in a pharmaceutical composition. The amount of the fatty acid ester of an estrogen or an estrogen derivative in the pharmaceutical composition of the present invention can range from about 0.01% to about 99% by weight of the pharmaceutical composition (“by weight”). The amount of fatty acid ester of an estrogen or an estrogen derivative can also range from about 0.1% to about 95% by weight, from about 0.1% to about 90% by weight, from about 0.1% to about 85% by weight, from about 0.1% to about 80% by weight, from about 0.1% to about 75% by weight, from about 0.1% to about 70% by weight, from about 0.1% to about 65% by weight, from about 0.1% to about 60% by weight, from about 0.1% to about 55% by weight, from about 0.1% to about 50% by weight, from about 0.1% to about 45% by weight, from about 0.1% to about 40% by weight, from about 0.1% to about 35% by weight, from about 0.1% to about 30% by weight. In certain embodiments, the fatty acid ester of an estrogen or an estrogen derivative can comprise about 1% to about 25% by weight or about 1% to about 20% by weight. In one embodiment, the percent of the fatty acid ester of an estrogen or an estrogen derivative is about 1% to about 15% by weight.

The pharmaceutical compositions used in the methods of the present invention can further include at least one pharmaceutically acceptable excipient. As used herein, the term “pharmaceutically acceptable” means suitable for use in, or in preparing a pharmaceutical product or pharmaceutical composition. The term “excipient” refers to a diluent, adjuvant (e.g., Freund's adjuvant (complete and incomplete)), filler, carrier, or vehicle with which the therapeutic is administered.

Such pharmaceutically acceptable excipients include, but are not limited to, water, salt solutions, alcohol, silicone, waxes, oils, polyethylene glycols, propylene glycol, liposomes, sugars, gelatin, surfactants, celluloses, polyvinylpyrollidones, or a combination thereof.

In certain embodiments, the pharmaceutical compositions of the present invention include an oil. Preferably, oils used in the present invention are edible. Oils suitable for the present invention can be triglycerides; such as, medium chain triglycerides, Labrasol®, Labrafil® M 125CS, Labrafac® CC, Captex® 300-6C and Captex® 300 EP; fats; oils derived from animal or plant oils; such as vegetable and fruit oils. Examples of such oils derived from plants include without limitation, canola oil; safflower oil; soybean oil; corn oil; olive oil; sesame oil; rapeseed oil; sunflower oil; rice oil; palm oil; cottonseed oil; linseed oil; tung oil; oiticica oil; castor oil; perilla oil; coconut oil, and blends thereof. The preferred oils of the present invention include canola oil, safflower oil, soybean oil, corn oil, olive oil, sesame oil and the medium chain triglycerides Captex® 300-6C, Captex® 300 EP.

Oils derived from animals and mineral oils may also be used in the present invention. Examples of such oils derived from animals include without limitation, fats such as tallow, lard and stearic acid, fish oils, fish-liver oils, oleic acid and sperm oil. Examples of mineral oils include without limitation, petroleum oils which are aliphatic, wax, aromatic, asphalt or mixtures thereof.

The amount of oil used depends on various factors, such as the type of oil used, the amount of fatty acid ester of an estrogen or an estrogen derivative used and the type of dosage form used. The preferred amount of oil is the amount that is required to suitably dissolve the fatty acid ester of an estrogen or an estrogen derivative. The solubility of a fatty acid ester of an estrogen or an estrogen derivative, such as oleoyl-estrone, in an oil such as, safflower oil, in terms of milligrams of the fatty acid ester in grams of oil can be between about 0.001 mg/g to about 500 mg/g when the solute is gravimetrically measured. In some embodiments the solubility is at least 25 mg/g, 50 mg/g or 200 mg/g when the solute is measured gravimetrically.

The amount of oil can range from about 1% to about 99% by weight of the pharmaceutical composition (“by weight”). The amount of oil can also range from about 5% to about 99% by weight, from about 10% to about 99% by weight, from about 15% to about 99% by weight, from about 20% to about 99% by weight, from about 25% to about 99% by weight, from about 30% to about 99% by weight, from about 35% to about 99% by weight, from about 40% to about 99% by weight, from about 45% to about 99% by weight, from about 50% to about 99% by weight. In certain embodiments, the oil can comprise about 55% to about 99% by weight, from about 60% to about 99% by weight, from about 65% to about 99% by weight, from about 70% to about 99% by weight, from about 75% to about 99% by weight, from about 80% to about 99% by weight, from about 85% to about 99% by weight, from about 90% to about 99% by weight, from about 95% to about 99% by weight.

The pharmaceutical compositions can take the form of solutions, suspensions, emulsions, tablets, pills, capsules, powders, sustained-release formulations and the like, depending on its intended route of administration. In a preferred embodiment, the pharmaceutical composition of the present invention comprises a fatty acid ester of an estrogen or an estrogen derivative in an oil, wherein the pharmaceutical composition is contained in a capsule. Preferred types of capsules include, but are not limited to, Capsugel, Licaps or Type 0 capsules. When the pharmaceutical composition of the present invention is formulated as capsules, the capsules can further be secured by any methods known in the art including sealing and banding methods. In certain embodiments during the sealing and banding process, preferred excipients include ethyl alcohol, polysorbate, gelatin and water.

Suitable routes of administration include parenteral (e.g., subcutaneous, intramuscular, intraorbital, intracapsular, intraspinal, intrasternal, intravenous, intradermal, intraperitoneal, intraportal, intra-arterial, intrathecal, transmucosal, intra-articular, and intrapleural,), transdermal (i.e., topical), epidural, and mucosal (e.g., intranasal) injection or infusion, as well as oral, inhalation, pulmonary, and rectal administration.

For oral administration, the pharmaceutical composition can include excipients such as sugars, e.g., lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrollidone (PVP); fats, oils, granulating agents; and binding agents such as microcrystalline cellulose, gum tragacanth or gelatin; disintegrating agents, such as cross-linked polyvinylpyrollidone, agar, alginic acid or a salt thereof such as sodium alginate, Primogel, or corn starch; lubricants, such as magnesium stearate or Sterotes; glidants, such as colloidal silicon dioxide; a sweetening agent, such as sucrose or saccharin; or flavoring agents, such as peppermint, methyl salicylate, or orange flavoring. If desired, solid dosage forms may be sugar-coated or enteric-coated using standard techniques.

For parenteral administrations, the pharmaceutical composition can comprise one or more of the following components: a sterile diluent such as water for injection, saline solution, fixed oils, polyethylene glycols, glycerol, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Also for parenteral administrations, the pharmaceutical compositions may be formulated in solutions, preferably in physiologically compatible buffers such as Hanks's solution, Ringer's solution, or physiological saline buffer. The solution may contain formulatory agents such as suspending, stabilizing and/or dispersing agents. In a preferred embodiment, the pharmaceutical compositions are formulated in sterile solutions.

Specifically for intravenous administration, suitable excipients include physiological saline, bacteriostatic water, Cremophor® EL (BASF; Parsippany, N.J.) or phosphate buffered saline (PBS). The excipient can also be a solvent or dispersion medium containing, for example, water, ethanol, polyol (for example, glycerol, propylene glycol, and liquid polyethylene glycol, and the like), and suitable mixtures thereof. In all cases, the pharmaceutical composition must be sterile and should be injectable through a syringe. The pharmaceutical compositions should be stable under the conditions of manufacture and storage and preserved against the contaminating action of microorganisms such as bacteria and fungi. The proper fluidity can be maintained by the maintenance of the required particle size by the use of a coating in the case of dispersion and by the use of surfactants in the case of a solution. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the pharmaceutical composition. Prolonged absorption of the injectable pharmaceutical compositions can be brought about by including in the pharmaceutical composition an agent which delays absorption, for example, aluminum monostearate and gelatin.

For transmucosal administration, penetrants appropriate to the barrier to be permeated are used in the formulation. Such penetrants are generally known in the art for transmucosal administration and include, but are not limited to, detergents, bile salts, and fusidic acid derivatives. Transmucosal administration can be accomplished through the use of nasal sprays or suppositories.

In addition to the formulations described previously, the pharmaceutical composition may also be formulated as a depot preparation. Such long acting formulations may be administered by implantation (for example subcutaneously or intramuscularly) or by intramuscular injection. Thus, for example, the therapeutic agents may be formulated with suitable polymeric or hydrophobic materials (for example as a solution or emulsion in an acceptable oil) or ion exchange resins, or as sparingly soluble derivatives, for example, as a sparingly soluble salt.

Additionally, the pharmaceutical composition can be delivered using a sustained-release system, such as semi-permeable matrices of solid polymers containing the pharmaceutical composition. Various forms of sustained-release materials have been established and are well known by those skilled in the art. Sustained-release capsules may, depending on their chemical nature, release the pharmaceutical composition for a few hours, days, weeks, months, up to over 100 days. Depending on the chemical nature and the biological stability of the pharmaceutical composition, additional strategies for stabilization may be employed.

The pharmaceutical compositions of the present invention can also be incorporated into liposomes. Such pharmaceutical compositions can be incorporated into liposomes using known techniques. In one embodiment, the pharmaceutical compositions of the present invention can be formulated a lipidic suspension. Such lipidic suspensions include lipoprotein suspensions and liposome suspensions, preferably obtained by addition of soy oil and egg phospholipids. The formulation should be substantially isotonic with the blood of the treated subject, and it should contain the pharmaceutical composition in the form of a stable lipidic suspension, i.e., in the form of finally divided particles incorporated in suspended microdrops with protecting layers of lipids, these lipids being of lipoproteins or of any common constituents of liposomes.

### Examples

A two-part study was conducted to evaluate the safety, tolerability, and pharmacokinetic (PK) profiles of single and multiple doses of oleoyl-estrone in humans.

### Part 1: Methods

For the first part of the study, thirty-six subjects where divided into groups of six. Each group was dosed with a single dose of 1, 3, 10, 30, 100, or 150 mg of oleoyl-estrone. Each individual in the group of 6 was randomized to oleoyl-estrone or placebo in a ratio of 2:1 where for every two individuals given oleoyl-estrone one is given a placebo.

Subjects under went a physical examination to establish height, weight, BMI and baseline signs and symptoms. Electrocardiograms and vital signs were taken. Blood for evaluation of hematology, coagulation and clinical chemistry and urine for urinalysis were obtained for each subject. Samples were also taken for hormone analysis, including estrone, estradiol and testosterone.

### Part 1: Results

The one-time single dose of oleoyl-estrone was safe, well tolerated at oral doses up to 150 mg and no serious adverse events were noted.

### Part 2: Methods

For the second part of the study, twenty four subjects were divided into groups of four. Each group was dosed with a single daily dose of 10, 30, 100, or 150 mg oleoyl-estrone for a seven day period. Each individual in the group of 4 was randomized to oleoyl-estrone or placebo.

Subjects under went a physical examination to establish height, weight, BMI and baseline signs and symptoms. Electrocardiograms and vital signs were taken. Blood for evaluation of hematology, coagulation and clinical chemistry and urine for urinalysis were obtained for each subject. Samples were also taken for hormone analysis, including estrone, estradiol and testosterone. Food-intake and motivation-to-eat using visual analogue scale questionnaires were analyzed.

Males and post-menopausal females were recruited in both parts of the study. The trial protocol provided that patients should maintain a normal diet and level of activity, but required that patients abstain from consuming alcohol. At baseline, in the placebo group me mean patient weight was 100.5±4.9 kg and BMI 34.4±3.5 and in the oleoyl-estrone group the weight was 100.5±17.8 kg and the average BMI was 34±4.2. The results discussed below are from the second part of the study.

### Part 2: Results

Oleoyl-estrone was safe, well tolerated at oral doses up to 150 mg and no serious adverse events were noted.

In the placebo group the most common adverse effects were back pain (n=2/8) and headache (n=2/8). In the oleoyl-estrone treated group, the most common adverse effects were diarrhea (n=3/17), headache (n=3/17) and back pain (n=4/17). One volunteer dropped out for personal reasons and one had post-menopausal bleeding. A review of her history revealed a similar episode 6 months prior to dosing. There were no clinically significant changes in the physical examinations, vital signs, ECGs, coagulation or liver function tests.

### Hematology

An increase in white blood cell count was seen in Day 7 but returned to baseline by Day 15. Total mean cholesterol showed a decrease at Day 7 with 100-150 mg oleoyl-estrone. Total mean TAGs showed a dose-dependent increase at all doses and on all days. Mean HDL cholesterol increased and mean LDL cholesterol decreased with an increase in the HDL:LDL cholesterol ratio even at the lower doses. Mean glucose concentration fell in a reversible and dose dependent manner and was still low at Day 15.

### Hormone Levels

Mean estradiol and estrone levels were elevated while mean testosterone levels were suppressed in a reversible and dose dependent manner. Effects on LH were less clear-cut. However, both LH and FSH are highly pulsatile (LH>FSH) and a large amount of variability is expected. Both leptin and CRP increased in a dose-dependent manner, returning to baseline by Day 15.

### Pharmacokinetics

Plasma concentration of oleoyl-estrone did not increase with the administered dose level. AUC, Tₘₐₓ and T_{1/2} could not be calculated. However, these results may be due to the limited sensitivity of the assay used. The 100 mg group had the highest Cₘₐₓ values of 2.4 ng/ml on Day 1 and 3.2 ng/ml on Day 7. On Day 1, oleoyl-estrone levels ranged from ranged from 0.3 to 2.4 ng/ml. On Day 7, the plasma levels of oleoyl-estrone were relatively constant and ranged from 0.3 to 3.0 ng/ml. The absence of significantly elevated plasma concentrations of oleoyl-estrone on Day 7 relative to Day 1, indicate that there is no clear accumulation in plasma after daily oral dosing for 7 consecutive days. In addition, there were no clear differences between the sexes with respect to the plasma concentration-time profiles.

### Body Weight and Food Consumption

Weight loss was seen in both active and placebo groups at Day 15. However, weight loss was greater and maintained in oleoyl-estrone treated subjects at Day 28 compared to placebo. The VAS scores and loss of weight was more or less dose-dependent, with maximal effects in the 30 mg and 100 mg groups. Oleoyl-estrone consistently demonstrated reductions in feelings of hunger, satiety and prospective food consumption.

While the foregoing description may represent preferred embodiments of the present invention, it should be understood that various additions, modifications, and substitutions may be made therein without departing from the spirit and scope of the present invention. In particular, it will be clear to those skilled in the art that the present invention may be embodied in other specific forms, structures, arrangements, and proportions, and with other elements, materials, and components, without departing from the spirit or essential characteristics thereof. The presently disclosed embodiments are therefore to be considered in all respects as illustrative and not restrictive, the scope of the invention being indicated by the appended claims and not limited to the foregoing description. Furthermore, all references mentioned herein are incorporated by reference in their entirety for all purposes.

1. A method of treating hyperandrogenism and conditions relating to hyperandrogenism in a subject comprising administering a fatty acid ester of an estrogen or an estrogen derivative.

2. The method according to claim 1, wherein the fatty acid ester of an estrogen or an estrogen derivative comprises oleoyl-estrone.

3. The method according to claim 1, wherein the fatty acid comprises an oleic acid.

4. The method according to claim 1, wherein the fatty acid ester of an estrogen or an estrogen derivative is administered orally.

5. The method according to claim 1, wherein the fatty acid ester of an estrogen or an estrogen derivative is administered in an amount of about 0.000005 mg/kg/day to about 20 mg/kg/day.

6. The method according to claim 1, wherein the fatty acid ester of an estrogen or an estrogen derivative is administered in an amount of about 0.00005 mg/kg/day to about 10 mg/kg/day.

7. The method according to claim 1, wherein the fatty acid ester of estrogen or an estrogen derivative is administered in an amount of about 0.0005 mg/kg/day to about 2 mg/kg/day.

8. The method according to claim 1, wherein the fatty acid ester of an estrogen or an estrogen derivative is administered in an amount of about 0.001 mg/kg/day to about 1 mg/kg/day.

9. The method according to claim 1, wherein the fatty acid ester of an estrogen or an estrogen derivative is administered in the form of a pharmaceutical composition.

10. The method according to claim 1, wherein the fatty acid ester of an estrogen or an estrogen derivative comprises about 0.01% to about 99% by weight of the pharmaceutical composition.

11. The method according to claim 1, wherein the fatty acid ester of an estrogen or an estrogen derivative comprises about 1% to about 15% by weight of the pharmaceutical composition.

12. The method according to claim 1, wherein the pharmaceutical composition further comprises an oil.

13. The method according to claim 12, wherein the oil comprises safflower oil, canola oil or soybean oil.

14. The method according to claim 12, wherein the oil comprises about 25% to about 99% by weight of the pharmaceutical composition.

15. The method according to claim 12, wherein the oil comprises about 80% to about 99% by weight of the pharmaceutical composition.

16. The method according to claim 1, wherein the conditions associated with hyperandrogenism comprise ovarian, breast, endometrial and prostate cancer, as well as other conditions such as, polycystic ovary syndrome; Cushing Syndrome; HAIR-AN (hyperandrogenism, insulin resistance and acanthosis nigricans) Syndrome; hirsutism; hyperplasia, such as congenital adrenal hyperplasia; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; overweight; obesity, excess body and facial hair; and alopecia.

17. A method according to claim 16, wherein the condition associated with hyperandrogenism is polycystic ovary syndrome.

18. The method according to claim 17, wherein treating polycystic ovary syndrome comprises treating the conditions associated with polycystic ovary syndrome comprise anovulation; oligoamenorrhea; amenorrhea; hirsutism; infertility; increased risk of miscarriage; miscarriage, ovarian cysts; acne; increased insulin levels; insulin resistance; hyperlipidemia, including high levels of cholesterol, cholesterol esters compounds, phospholipids and triglycerides; impaired glucose tolerance; type-2 diabetes; cardiovascular disease; hypertension; virilism; Cushing Syndrome; excess body and facial hair; and alopecia.

19. A pharmaceutical composition described in claim 9 for treating hyperandrogenism and conditions associated with hyperandrogenism.

20. A composition according to claim 19, wherein the condition associated with hyperandrogenism is polycystic ovary syndrome.

21. Use of a fatty acid ester of an estrogen or an estrogen derivative described in claim 20 in the manufacture of a medicament for the treatment of hyperandrogenism and conditions associated with hyperandrogenism. manufacture of a medicament for the treatment of hyperandrogenism and conditions associated with hyperandrogenism.

22. Use according to claim 21, wherein the condition associated with hyperandrogenism is polycystic ovary syndrome.

23. A method of modulating the levels of androgens in a subject in need thereof comprising administering a fatty acid ester of an estrogen or an estrogen derivative described in claim 1.
